(19) United States
(12) Patent Application Publication
(10) Pub. No.: US 2025/0352109 A1
(43) Pub. Date: Nov. 20, 2025

(54) MEDICAL ELECTRODE

(71) Applicant: AMBU A/S, Ballerup (DK)

(72) Inventor: Frederik Nikolaj Sværke SONNENBORG, Jyllinge (DK)

(73) Assignee: AMBU A/S, Ballerup (DK)

(21) Appl. No.: 19/207,056

(22) Filed: May 13, 2025

(30) Foreign Application Priority Data
May 14, 2024 (EP) 24175725.1

(51) Int. Cl. A61B 5/265; A61B 5/055; A61B 5/266; A61B 5/273; A61B 5/291; A61B 5/30

(52) U.S. Cl. CPC A61B 5/265; A61B 5/055; A61B 5/266; A61B 5/273; A61B 5/291; A61B 5/303; A61B 2562/04; A61B 2562/125

(57) ABSTRACT

A medical electrode with a lead wire connector, the electrode including an electrode body with a skin side having a peripheral skin contact area and a central cavity with a central cavity area, both areas having an electrically conductive surface, the central cavity area being provided with at least one protrusion extending within the cavity only, and an outer side opposite to the skin side.

### CROSS-REFERENCE TO RELATED APPLICATIONS

This application claims priority from and the benefit of European Patent Application No. 24175725.1, filed May 14, 2024; the disclosure of said application is incorporated by reference herein in its entirety.

### TECHNICAL FIELD

The present technology relates to a medical electrode for measuring a biopotential, an electrode array comprising the medical electrode, and a method for manufacturing the electrode and electrode array.

### BACKGROUND

Electrodes are medical field devices used for patient check-ups and monitoring procedures in the health setting. Medical electrodes convey internal ionic current of the patient's body into electrical current that can be amplified and evaluated to determine various conditions, disorders or diagnosis. Some of the main types of health examinations utilizing medical electrodes include electrocardiograma evaluate electrical activity of the heart, electroencephalograhy (EEG), to measure electrical waves of neuronal activity of the brain, polysomnygraphy (PSG), and electrophysiology (EP). EEG is the measurement of electrical potential differences across points on the scalp. These electrical potential differences are the result of electrical activation of diverse brain areas and are associated with brain function. The coherent activity of cortical pyramidal neurons generates ionic currents and these, in turn, give rise to an electric field and scalp electric potential. EEG opens a window into the functioning brain because neural function relies on electrochemical communication. The electric fields generated by the cortex provide a powerful, direct measure of its processes via EEG, which can record specific brain wave patterns in the patient. Cup electrodes may be attached to the scalp of a patient to detect neuronal activity of the brain, which is measured in microvolts (μV).

Single-use electrodes optimize workflow and reduce cost while saving patient's lives and improving patient care. They optimize workflow and reduce cost because they are always ready when needed without the traditional large-scale capital and repair budgets required for reusable electrodes. For example, a sterilization and storage facility is avoided, there is no need to maintain evidence of sterilization, and there is no need to transport electrodes from sterilization and storage facilities to the buildings where they are needed, sometimes in the middle of the night or weekends. They save patient's lives and improve patient care because they are readily available and do not pose a cross-contamination risk. This also reduces hospital re-admissions. While single-use electrodes are disposed after a single patient use (one or more procedures may be performed while the patient remains in the treatment room), the environmental impact of re-useable electrodes, due to cleaning materials, CO₂ emissions during the cleaning process, and use of disposable personal protective equipment by personnel involved in transportation and sterilization of the re-useable electrodes, can be similar to that of single-use electrodes.

To further enhance the benefits of single-use medical electrodes, it is desirable to expand the applicability of the electrodes and to reduce manufacturing costs.

### SUMMARY

The present technology provides a medical electrode. A first aspect of the present technology relates to expanding the applicability of the electrodes. An embodiment of the first aspect relates to a medical electrode comprising: a lead wire connector, an electrode body including: a skin side; an outer side opposite the skin side; a central cavity having a height; and a rim surrounding the central cavity and having a thickness smaller than the height, the rim having a peripheral skin contact area and the central cavity having a central cavity area, each of the areas being on the skin side and having an electrically conductive surface, the central cavity area comprising at least one protrusion extending within the central cavity only. The protrusion increases the electrically conductive surface area, which increases the signal quality. Further the usable time of the electrode increases with the increased surface area. The usable time may be extended to a degree to enable long term monitoring ranging from a few hours to more than 24 hours, such as several days, e.g. 7 days. This may be advantageous when using the electrodes in an intensive care unit (ICU) setting and for examinations such as epilepsy and sleep studies, where monitoring for extended time is advantageous. Preparation of the patient, correct placement of the electrodes and correct set-up of the EEG system may take a trained health care professional considerable time, such as up to one hour, so replacement of electrodes is very time consuming and entails a risk that the electrodes are not positioned correctly. Enabling monitoring for extended periods without renewal of electrodes hence reduces the time and cost involved in providing EEG, so provision of an electrode usable for monitoring for extended time periods is advantageous.

The at least one protrusion may be at least one rib arranged with its longitudinal extent aligned substantially radially, such as in a radial direction +/−10 deg, relative to a central axis through the central cavity. The at least one rib is hence arranged substantially in a radial plane containing a central axis of the central cavity. Substantially radial direction is considered to include a deviation of +/−10 deg, e.g. having a direction comprising a tangential component. The at least one protrusion could have other shapes, such as the shape of a pin, more than one pin, pins aligned along the radial plane, an annular rib, sectors of a circle, or a combination of shapes.

As used herein, “in the range” includes the values that define the range. Therefore, “in the range of A-B” includes A and B.

The number of protrusions may be less than 10, such as in the range of 2-8, for instance 3-5. A high number of protrusions provides some benefits, such as the possibility of a comparatively larger surface area, as well as the possibility of improved grip to gel or adhesive. However, a high number of protrusions may increase manufacturing complexity and cost and may result in reduced filling of the cavity in case a gel is used, which may negatively influence the performance of the electrode. At present three protrusions are considered a suitable compromise.

The total active area of the electrode may be in the range of 95 to 130 mm², such as 100 to 110 mm², for example 102 to 105 mm². The total active area is defined as the area of the electrode taking part in picking up signals. The total active area is considered to include the skin contact area and rounded edges thereof, if any, the cavity surface area and the protrusion surface area.

The central cavity may have, approximately, the shape of a truncated cone, the dome may have a frustro-conical shape. The dome may have other shapes, such as a cylinder, a polygon prism or a cone. The truncated cone shape is, however, considered an advantageous compromise when considering ergonomics for the patient and maximizing active surface area.

The lead wire connector may comprise, prior to attachment of a conductor, a depression and at least one wall adjacent the depression. The depression may be sectioned by a step to provide a shallow conducting portion and a deep connection portion. The terms “deep” and “shallow” refer to the depth of the portion of the depression. The lead wire connector may be offset from a plane on which the skin contact area lies. The offset prevents heat generated at the lead wire connector, for example during an MR scan, from discomforting the patient.

The medical electrode may comprise polymers that provide rigidity to the medial electrode, such that when force is applied against the electrode the force presses the electrode to the patient substantially without deforming. While the rim may be thin enough to slightly deform, the dome will substantially retain its shape when the electrode is applied to the patient.

Monitoring in neurological ICU's is often combined with (unplanned) Magnetic Resonance Imaging (MRI) or Computed Tomography (CT) scans. MRI uses strong magnetic fields (in the Radio Frequency range) to generate images of organs in the body, whereas CT scans use X-rays. For MRI the strong magnetic field may lead to possible reactions to metals introduced into the MR scanner. Traditional medical electrodes and electrode arrays are MR unsafe and should be removed before any MR scan, as MR unsafe parts could introduce a risk to the patient because of excessive heating of the parts. Further, MR unsafe parts could deteriorate the imaging of MR or CT scans due to artifacts in the image resulting from the scan. Removal and refitting the electrodes is a tedious and time-consuming task. Providing a MR conditional electrode, meaning electrodes suitable for use in magnetic resonance (MR) scanning, and electrode array hence expand the applicability of such electrodes and electrode arrays.

The peripheral skin contact area may comprise cut-outs in its periphery, such as four cut-outs, with a depth in the range of 5-15% of the outer diameter of the peripheral skin contact area, such as 10% of the diameter, e.g. 0.5-1.5 mm, such as 1 mm in the case of a diameter of 10 mm. By depth is meant the radial extension of the cut-out from the periphery. The cut-outs may lower potential formation of eddy currents in the electrode during an MR scan and hence lower the potential risk of artifacts on MRI.

To further reduce environmental impact, the electrodes according to the present technology may be made, primarily, of polymer materials. The primary materials, polymers, may contain dopants in the chemical structure of the polymers, such as chemicals or additives, that enable charge movement. Alternatively, conductive fillers may be added in a composition formed primarily, for example at least 90% by weight, of polymers. Example fillers include carbon nanotubes and graphene. The addition of conductive fillers creates a conductive composite. To reduce costs, the electrode may comprise a multilayer structure in which a skin facing layer contains the dopants or fillers, collectively referred to as “conductivity enhancing agents.” In other words, the material consists substantially of polymers and contains conductivity enhancing agents. The material from which the electrode is made may be devoid of metals other than the conductivity enhancing agents.

The electrode body may comprise a polymer based core material with a conductive surface coating of Ag/AgCl. Hereby it is possible to provide an electrode with a relatively low environmental impact in that the electrode contains very low amounts of metal, which is considered to have a relatively high environmental impact. Further, such an electrode is suitable for mass production at relatively low cost, and hence particularly relevant for single-patient use. The low amount of metal also reduces the potential risk of artifacts in MR or CT scans.

The cavity may be prefilled with a conductive gel. Providing an electrode with prefilled cavity may simplify work for the health care specialist in that the electrode is ready to use without any further preparation and is hence time saving. Downside is that the shelf life of the electrode may become shorter, so it is not always an advantage. Further the health care specialist may prefer a specific adhesive or gel for any given task, and in this case it is a disadvantage if the electrode is prefilled with another gel.

Another aspect of the present technology relates to an electrode array comprising a plurality of medical electrodes according to the first aspect and a plurality of lead wires each comprising a lead wire conductor with an electrically insulating cladding, where each lead wire has a first end physically embedded in the lead wire connector and electrically connected to the electrically conducting surface of the electrode, and a second end connected to a connector. Hereby an electrode array may be provided having a very low amount of metal to the benefit of sustainability, and also lowering the risk of safety of image quality issues in MR or CT scans.

The lead wire conductor may be made of an electrically conductive non-magnetic material, such as carbon or copper. Using a non-magnetic material lowers the risk of safety or image quality issues in MR or CT scans.

The length of lead wire may be in the interval of 30 to 50 cm, such as 35 to 45 cm, such as approximately 40 cm. Such a lead wire length has hitherto been considered too long for MR conditional arrays, where standard lead wire length is approximately 25 cm. The lead wire may act as antenna in the magnetic field and cause heating, but the present inventor has found that it is a technical prejudice that the lead wire should have a length of 25 cm or less, and that a lead wire length in the given interval may indeed be considered MR conditional. A short lead wire length of e.g. 25 cm makes it difficult to correctly position the electrodes, and further, the connector at the second end may in this case end up in a position which is unpleasant for the patient. Also, the relatively short lead wire length leaves very little room for adjustment of the connector position. It is not excluded, however, to use lead wires shorter that 40 cm, or shorter than 25 cm, to capitalize on other features of the technology disclosed herein.

Another aspect of the present technology relates to a method of making an electrode array as discussed above, the method comprising: providing an electrode body with a lead wire connector; positioning a lead wire in the lead wire connector; applying energy to material of the lead wire connector to soften or melt the material; and allowing the softened or molten material to set around the lead wire, thereby integrating the lead wire in the lead wire connector of the medical electrode. This method simplifies the manufacturing process and reduces the manufacturing cost. Further with this method there is no need for mechanical anchoring or fixation by use of a metal crimp or anchoring and hence the risk of image artifacts in MR or CT scans is reduced.

One or more the aforementioned objects may be met by aspects of the present technology described in the following embodiments, variations and examples thereof.

A person skilled in the art will appreciate that any one or more of the above aspects of the present technology and embodiments thereof may be combined with any one or more of the other aspects and embodiments thereof.

### BRIEF DESCRIPTION OF THE DRAWINGS

The present technology will be described in more detail below with reference to the following figures. The figures illustrate embodiments, variations and examples of the present technology to facilitate the understanding of a person of ordinary skill in the art and are not to be construed as limiting the scope of the claims.

FIG. 1 is a perspective illustration of an EEG system system including an electrode array, an electrode harness, a connector box and a computer;

FIG. 2 is an illustration of electrodes as applied to a patient,

FIG. 3 is a view from below of an embodiment of an electrode;

FIG. 4 is a top view of the electrode of FIG. 3;

FIG. 5 is a perspective top view of the electrode of FIG. 3,

FIG. 6 is a sectional side view of the electrode of FIG. 3;

FIG. 7A is a detail of FIG. 6 including a lead wire and a tool;

FIG. 7B corresponds to FIG. 7A with the lead wire integrated;

FIG. 8 is a perspective view from below of the electrode of FIG. 3,

FIG. 9 is a sectional side view of the electrode of FIG. 3 as attached to a patient;

FIG. 10 is a top view of the electrode of FIG. 3 with additional features;

FIG. 11 is a sectional side view of the electrode of FIG. 10;

FIG. 12 is a flowchart illustrating a method of making a medical electrode; and

FIG. 13 is a flowchart illustrating a method of making a medical electrode array.

### DETAILED DESCRIPTION

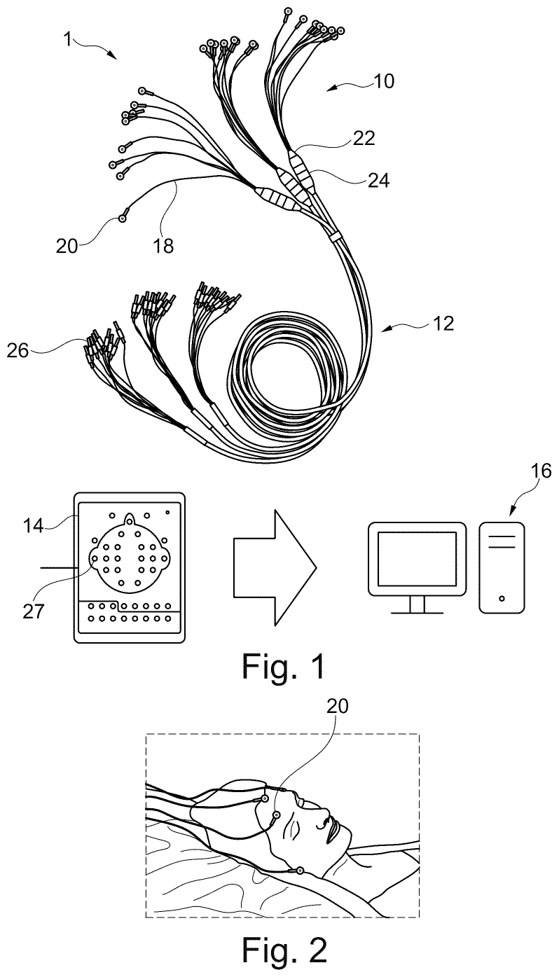

FIG. 1 illustrates an embodiment of an EEG system 1 including an electrode array 10, a harness 12, a connector box 14 and a processor 16. The electrode array 10 comprises several lead wires 18 each having a first end with an electrode 20 and a second end with an electrode connector 22. The harness 12 similarly has a first end with a harness connector 24 connectable to the electrode connector 22, and a second end with connectors, such as the illustrated harness jacks 26. The harness jacks 26 are in turn adapted to connect to the connector box 14 via sockets 27. The connector box in turn is adapted to connect to the processor 16 as schematically illustrated by an arrow. The connector box 14 and the processor 16 may be separate parts but could, alternatively, be provided as one unit. The processor 16 is adapted to process input from the electrodes 20 and output a signal that can be presented to a physician, such as in a graph on a screen or on print. The array 10 may be magnetic resonance (MR) conditional, meaning that under certain conditions it is safe to include the array 10 in an MR scan without a risk of harm to the patient and with low risk of negatively influencing image quality of the MR scan. For this the array 10 may be disconnected from the harness 12, which may be a reusable, MR unsafe part to lower cost and environmental impact, and in this event the harness 12 should not enter the MR room.

Electrodes as applied to a patient are illustrated in FIG. 2. The electrodes 20 are attached to the head of the patient, such as the scalp and forehead. Prior to application of the electrodes the skin of the patient is typically prepared to reduce the electrical resistance, such as by abrading the skin of the patient at the positions for electrode application. A gel, paste or adhesive may be used to attach the electrodes to the head of the patient. The gel, paste or adhesive may be electrically conductive, e.g. by containing chloride ions. As an example, Ten20(RM) Conductive Paste, by Weaver and Company, may be used. It is also possible to use the electrodes in dry state, without a gel, or to use e.g. a wet sponge with a saline solution. However, an electrically conductive gel is generally considered advantageous and may improve conduction and reduce skin-electrode interface impedance. Therefore, the gel between the skin and electrode allows for good-quality recording of biopotentials, which is measured in μV. Supplementary or alternatively, tape may be used to attach the electrodes to the head of the patient. In some cases, an elastic electrode cap is used. An advantage of the elastic electrode cap is that application of the electrodes to the patient can be done very quickly. A disadvantage of the elastic electrode cap is that it may feel restrictive to the patient as the cap generally needs to be held down with straps anchored around the chin or chest. Further, such caps are generally not for single patient use and must hence be cleaned and dried after each use. Additional individual electrodes come at a lower cost and a versatile application area.

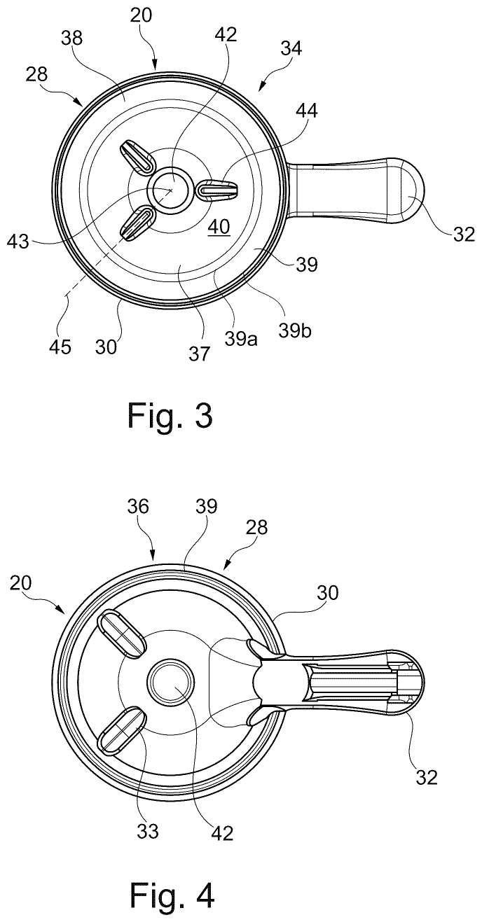

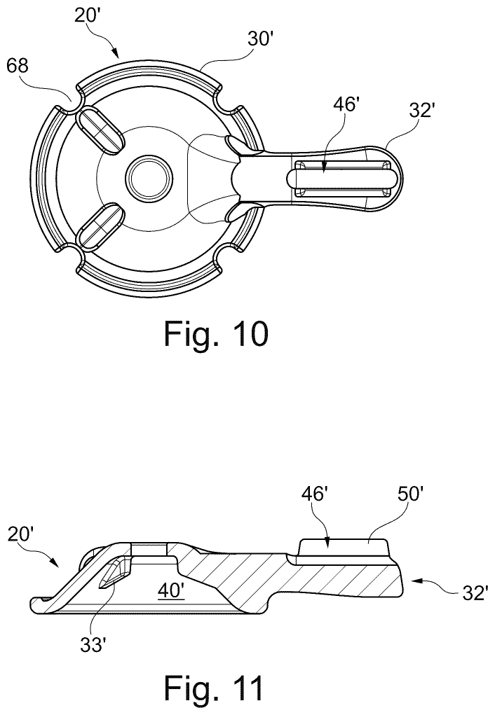

An embodiment of the electrode 20 is shown in FIGS. 3 to 9. The electrode body 28 has a skin side 34, illustrated in FIG. 3, adapted to face the skin of a patient, and an outer side 36, illustrated in FIG. 4, opposite the skin side. Turning first to a view from the skin side, the illustrated electrode 20 has an electrode body 28 with a periphery 30 and a lead wire connector portion 32. As shown, the periphery 30 is circular. The periphery 30 also comprise arcs of a circle separated by cut-outs, as shown in FIG. 10, or other shapes such as oval. The lead wire connector portion 32 is adapted to connect to the lead wire 18, as will be discussed in more detail below with reference to FIGS. 7A and 7B. The lead wire connector portion 32 may double as a mini-handle for the health care professional to ease handling and positioning of the electrode 20.

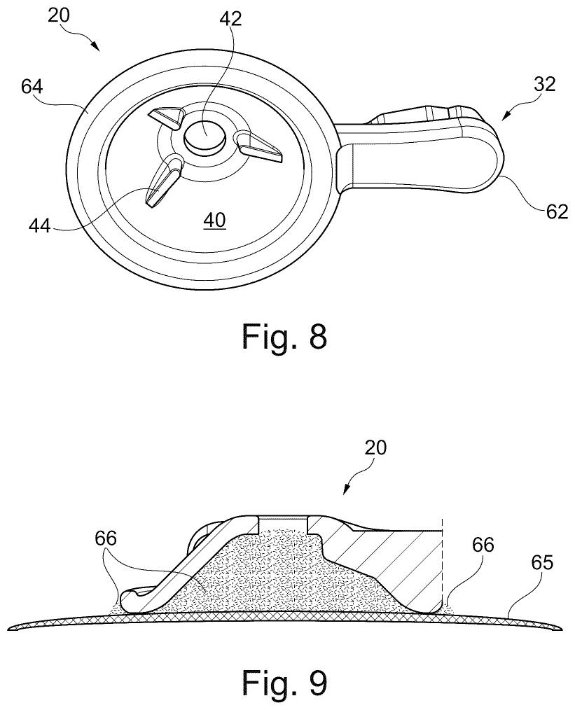

The electrode body 18 comprises a dome 37 and a rim 39 extending radially outwardly from the dome 37 and surrounding a central cavity 40 formed by the dome 37. Most of the dome is comprised by a wall that forms the central cavity 40, which therefore also has the shape of the dome 37. Therefore, as used herein, the term “dome” refers to a central portion that extends outwardly from the rim without regard for its shape. The rim 39 has a peripheral skin contact area 38 extending between a rim inner edge 39a and a rim outer edge 39b of the rim 39. The central cavity 40 extends longitudinally outwardly from the skin contact area 38 such that a height of the central cavity 40 is greater than a thickness of the rim 39. The central cavity 40 has the approximate shape of a dome or truncated cone (see for example FIGS. 6 and 8). The central cavity 40 may, as illustrated, have an opening 42. The opening 42 allows for needle insertion and escape of air or surplus gel, if any. As an example, the opening may have a diameter in the interval of 1.5 to 2 mm. A central axis 43 intersects the opening 42 and extends through the central cavity 40, as also indicated on FIG. 6. Protrusions 44, here in the shape of ribs, extend inwardly from an inner surface of the dome 37 inside the central cavity 40 (see FIGS. 3, 6 and 8). The protrusions, or ribs, 44 may be arranged in radial planes 45. The ribs 44 increase the surface area of the central cavity's interior, and further may assist in holding any gel or paste in the central cavity 40. The central cavity 40 of the electrode 20 may be prefilled with a gel or paste, thereby providing a ready-to-use electrode. Alternatively, the electrode 20 may be supplied without gel or paste, which may be added to the electrode at a later stage, such as the time of application to the patient. The skin contact area 38 and the dome 37 have electrically conductive surfaces. The electrode may or may not be made of, or comprise, a conductive material, whereas the surfaces should be conductive. The conductive surface may be provided as a coating, a lamination, or a layer of the body, for example. The lamination layer or the coating may be applied in a mold so that they bond with the polymer that forms the body. The lamination layer or the coating may be applied to the body post-molding. The layer of the body may be injection-molded in a two-material or two-step process. The electrically conductive surface may comprise silver/silver chloride (Ag/AgCl), which is found to provide consistent and superior signal quality. Increasing the surface area improves signal quality and enables longer working periods of the electrodes. The ribs/protrusions are sized and positioned so that they are contained inside the interior of the central cavity 40. As the ribs do not extend outside of the interior of the central cavity, the ribs do touch the skin of the patient and hence do not negatively influence the patient's comfort.

For optimum patient comfort the peripheral skin contact area 38 preferably has curvatures having radius of at least 0.3 mm, such as at least 0.5 mm, at the transition from the skin contact area 38 to the interior cavity 40 at the inner periphery, e.g. from the inner rim edge 39a, and similarly at the outer periphery of the skin contact area 38, e.g. from the outer rim edge 39b. This is primarily of relevance for electrodes positioned on a patient's head, where there is a risk that the electrode will be squeezed between the patient and a substrate. A height hₑ (shown in FIG. 6) of the electrode in the interval of 2.5 to 3.5 mm, such as 2.8 to 3.2, or such as 3 mm is found to provide a reasonable compromise between patient comfort and signal quality.

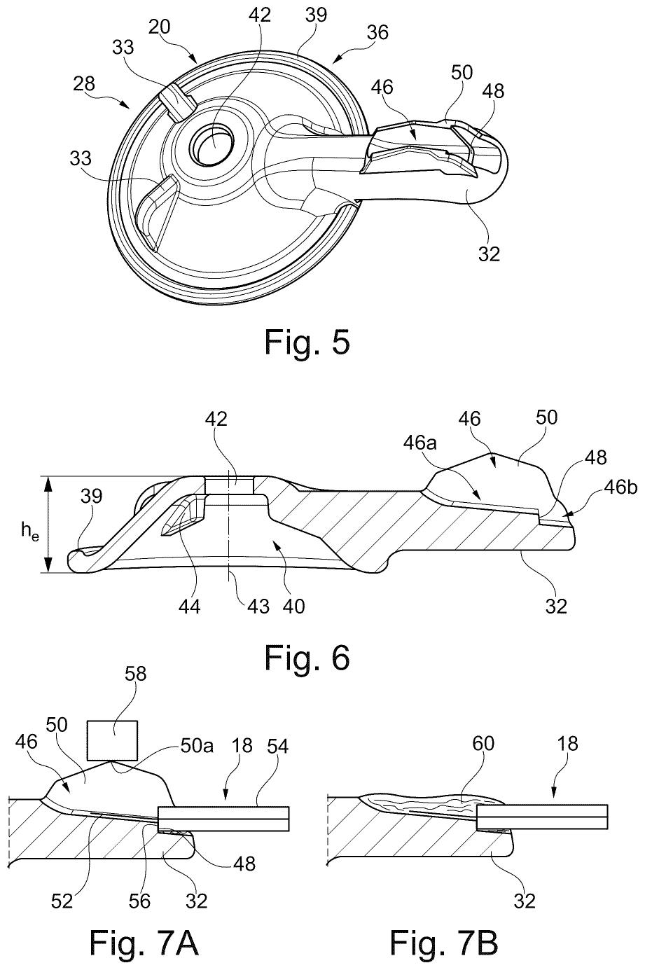

Referring now to FIG. 4, two fixation wings 33 are provided on the electrode body 28 for easier positioning (see also FIG. 5). The diameter of the electrode body in this embodiment is 10 mm, which is found to provide a reasonable compromise between size of the electrode and signal quality. If the electrode body has a much larger diameter it may be difficult to place electrodes correctly, especially when applied to children, whereas if the electrode is much smaller signal quality may become poor, and small electrodes may be difficult to handle for a health care professional.

FIGS. 5 and 6 illustrate features of the lead wire connector 32. The perspective top view of FIG. 5 illustrates the electrode 20 and specifically them second, or outer, side 36 thereof with the wings 33, the opening 42 and the lead wire connector 32. FIG. 6 is a sectioned side view of the electrode 20. The lead wire connector 32 comprises a depression 46 comprising a step 48 as also illustrated in FIG. 6. The depression is bordered at two sides by walls 50. The step 48 sections the depression 46 into a shallow conducting portion 46a and a deep connection portion 46b. As used herein, deep and shallow refer to the depth of the recess relative to an outer edge of the wall 50. The height hₑ of the electrode 20, described above, is also shown. Further, FIG. 6 illustrates that the ribs 44 may be positioned at the same radial position as the wings 33 and the lead wire connector 32. Superimposing the positions may be facilitate molding of the electrode 20 in view of flow of material in the mold. By “superimposing” it is meant that a radial plane traverses a rib or protrusion 44 and a wing 33, or a rib or protrusion 44 and the lead wire connector 32.

The lead wire connector 32 serves as a handle which the medical practitioner can use to place the electrode. In the magnetic field in the MR scanner the lead wire will act as an antenna and take up energy. The connection between the lead wire and the lead wire connector is achieved by e.g. ultrasonic welding, where material of the electrode will flow and surround the lead wire. The body has an electrically conducting surface coating and further may comprise material (polymer) with conductivity agents e.g. particles. The electrical connection between body and the lead wire will be a weld zone with a mixture of conducting surface coating and polymer material with conducting particles/conductivity agents. The resulting electrical connection may have some electrical resistance greater than the electrical resistance of the lead wire. The electrical connection may generate heat, due to the electrical resistance, beneficially in a part of the electrode that does not contact the patient. The lead wire connector 32 has some distance to the skin, and further a portion of the lead wire connector 32 (mainly the wire connection/weld zone) may be covered by a heat shrink tube to provide mechanical strength and thermal protection for the skin of the patient in the case the patient undergoes an MRI scan.

FIGS. 7A and 7B are sectional views of the lead wire connector 32 in a process to connect a lead wire to the lead wire connector 32 of the electrode. FIG. 7A schematically illustrates a lead wire 18 with a lead wire conductor 52 having an insulating cladding 54. A portion of the lead wire 18 is stripped and the bare lead wire conductor 52 positioned in the conducting portion 46a of the depression 46 and with an unstripped portion of the lead wire positioned in the connection portion 46b with an end 56 of the insulating cladding positioned against the step 48. On top of the wall a tool 58 may be applied, such as an ultrasonic horn or a heat staking tool, to at least partially soften or melt the walls 50. As shown, the wall 50 comprises an optional peak 50a, which may aid in focusing the energy of the tool 58. The material of the walls redistribute as embedding material 60 to embed the lead wire in the lead wire connector 32 and electrically connect the lead wire conductor 52 to the electrode. This allows for automation and provides a secure connection without adding material. Other ways to connect the lead wire to the electrode are conceivable, such as using a crimp bushing or by soldering, which are simple and well known ways of connecting wires. However, these alternatives are less advantageous as they have some drawbacks. For example, the use of crimp bushings may result in faulty connection if the parts are not carefully arranged prior to application. Further, the electrical contact is not always optimum. Finally, such bushings are generally made of metal, which should be avoided for MR conditional electrodes. In case of soldering, cold solder joints may occur, creating an incorrect joint. Cold solder joints are not always easy to detect and may result in faulty electrodes. Further, the use of solder adds metal to the electrode, which should be avoided for MR conditional electrodes.

FIG. 8 provides a view of the skin, or first, side of the electrode showing the interior cavity 40, the opening 42 and the ribs 44. The lead wire connector 32 advantageously has a rounded free end 62 to limit any potential discomfort for a patient should the lead wire connector 32 get in contact with the skin of the patient. Similarly, the outer edge 64 of the electrode 20 preferably has a rounded profile to minimize any potential discomfort for the patient.

FIG. 9 schematically illustrates the electrode's position on the skin 65 of a patient. Here a conductive gel 66 is applied to the electrode to attach the electrode to the skin 65, and to lower the skin-electrode interface impedance.

Additional features, which are optional, of the electrode 20, here denoted as electrode 20′, are illustrated in FIGS. 10 and 11. The electrode 20′ shares many features of the electrode 20, such as the dome 37 and the rim 39, but it also has some differences, which will be discussed in the following. When comparing FIG. 10 and FIG. 4, it can be seen that the electrode 20′ comprises cut-outs 68 in its periphery. The cut-outs 68 minimize the potential formation of eddy currents in the electrode 20′ when subjected to a changing magnetic field, such in an MR scanner. Eddy currents could potentially lead to artifacts on the MR image, so with the cut-outs 68 an electrode 20′ is provided with reduced risk of artifacts in MR imaging. The diameter of the electrode 20′ could be around 10 mm. The risk of eddy current formation and artifacts increases with increasing diameter, so the beneficial effect of providing the cut-outs 68 will be more pronounced for larger electrodes. Here, a total of four cut-outs are provided, but one cut-out 68 could in some cases be enough, e.g. for electrodes having a small diameter, or if there is no specific need for artifact-free images. If more than one cut-out is provided, the cut-outs should preferably be equidistant from each other in a radial direction to provide the shortest possible unbroken section of the periphery. Having more than five cut-outs is generally not recommended for the electrodes as the active area of the electrode is considered to be reduced too much, and further provision of cut-outs complicates molding. The illustrated cut-outs are rounded but could alternatively be slots or V-shaped cut-outs. Rounded cut-outs are, however, presently preferred to limit the risk of sharp edges, which could give rise to discomfort for the patient. The peripheral cut-outs 68 can also be provided in the electrode 20 shown in FIGS. 3 to 7B. The cut-outs 68 may extend past the rim outer edge 39b into the rim 39.

Another difference relates to the lead wire connector, which can be seen when comparing the sectional view of FIGS. 6 and 11. The lead wire connector 32′ of the electrode 20′ comprises a simplified wall 50′ having a straight upper edge without a peak, making the electrode slightly simpler to produce. A lead wire (not shown) may be positioned in the depression 46′ and embedded in material in a similar way as exemplified above. As shown, the step 48 is omitted. In another example of the electrode 20′, the step 48 is provided. Further, the lead wire connector 32′ can be provided in the embodiment of the electrode 20 shown in FIGS. 3 to 7B.

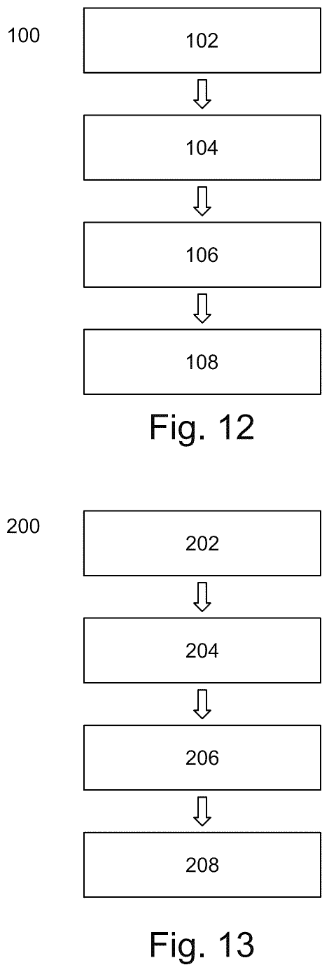

Another aspect of the present technology relates to a method of making a medical electrode as discussed above, which will now be described with reference to a flowchart 100 in FIG. 12. In an embodiment of the method of making medical electrodes, the method comprises: at 102, providing a mold with protrusion forming recesses; at 104, injecting a polymer into the mold; at 106, ejecting the electrode blank from the mold; and at 108 coating the electrode blank with an electrically conductive surface layer, such as Ag/AgCl. The polymer may for example be polycarbonate (PC) or Acrylonitrile Butadiene Styrene (ABS) or a mixture of PC and ABS, potentially with reinforcing fibers, such as carbon fibers.

A further aspect of the present technology relates to a method of making an electrode array as discussed above, which will now be described with reference to a flowchart 200 in FIG. 13. In an embodiment of the method of making medical electrodes, the method comprises: at 202, providing an electrode body with a lead wire connector; at 204, positioning a lead wire in the lead wire connector; at 206, applying energy to material of the lead wire connector to soften or melt the material; at 208, allowing the softened or molten material to set around the lead wire, thereby integrating the lead wire in the lead wire connector of the medical electrode.

The following items are examples of various embodiments disclosed above:
1. A medical electrode, comprising: a lead wire connector, and an electrode body including: a skin side; an outer side opposite the skin side; a central cavity having a height; and a rim surrounding the central cavity and having a thickness smaller than the height, the rim having a peripheral skin contact area and the central cavity having a central cavity area, each of the areas being on the skin side and having an electrically conductive surface, the central cavity area comprising at least one protrusion extending within the central cavity only.
2. Medical electrode of item 1, wherein the at least one protrusion is at least one rib arranged substantially radially, such as radial direction +/−10 deg, relatively to a central axis through the central cavity.
3. Medical electrode of any one of the items above, wherein the number of protrusions is below 10, such as in the range 2-8, for instance 3-5.
4. Medical electrode of any one of the items above, wherein the electrode has a total active area in the range of 95 to 130 mm², such as 100 to 110 mm², for example 102 to 105 mm².
5. Medical electrode of any one of the items above, wherein the central cavity approximately has the shape of a dome or truncated cone.
6. Medical electrode of any one of the items above, wherein the lead wire connector comprises a depression and at least one wall adjacent the depression.
7. Medical electrode of item 6, the depression is sectioned by a step providing a shallow conducting portion and a deeper connection portion.
8. Medical electrode of any one of the items above, wherein the peripheral skin contact area comprises cut-outs in periphery (clover), such as four cut-outs, each having a depth in the range of 0.5-1.5 mm, such as 1 mm.
9. Medical electrode of any one of the items above, wherein the body comprises a polymer-based core material with a conductive surface coating of Ag/AgCl.
10. Medical electrode of any of the items above, wherein the cavity is prefilled with a conductive gel.
11. Electrode array comprising a plurality of medical electrodes of item 1 and a plurality of lead wires each comprising a lead wire conductor with an electrically insulating cladding, where each lead wire has a first end physically embedded in the lead wire connector and electrically connected to the electrically conducting surface of the electrode, and a second end connected to an electrode connector.
12. Electrode array of item 11, wherein the lead wire conductor of each lead wire is made of an electrically conductive non-magnetic material, such as carbon or copper.
13. Electrode array of item 11 or 12, wherein each lead wire has a length in the interval of 30 to 50 cm, such as 35 to 45 cm, such as approximately 40 cm.
14. A method of making an electrode array, the method comprising: providing an electrode body with a lead wire connector; positioning a lead wire in the lead wire connector; applying energy to material of the lead wire connector to soften or melt the material; allowing the softened or molten material to set around the lead wire, thereby integrating the lead wire in the lead wire connector of the medical electrode.

The use of the terms “first”, “second”, “third”, “fourth”, “primary”, “secondary”, “tertiary” etc. does not imply any particular order or importance. These labels are included to identify individual elements. Furthermore, the labelling of a first element does not imply the presence of a second element and vice versa.

### PARTS LIST

1 EEG system
10 EEG array
12 harness
14 connector box
16 processor
18 lead wire
20, 20′ electrode
22 electrode connector
24 harness connector
26 jack
27 socket
28 electrode body
30 periphery
32 lead wire connector
33 fixation wing
34 skin side
36 outer side
37 dome
38 skin contact area
39 rim
39a rim inner edge
39b rim outer edge
40 central cavity
42 opening
43 central axis
44 rib
45 radial plane
46 depression
46a conducting portion
46b connection portion
48 step
50 wall
52 lead wire conductor
54 insulating cladding
56 end
58 tool
60 embedding material
62 rounded end
64 outer edge
65 skin
66 gel
68 cut-out
100 flowchart
200 flow-chart
hₑ electrode height

We claim:

1. A medical electrode, comprising:
an electrode body including:
a skin side;
an outer side opposite the skin side;
a dome comprising a central cavity and having a height, the central cavity comprising a central cavity area and at least one protrusion extending only within the central cavity; and
a rim surrounding the central cavity and having a thickness smaller than the height, the rim having a skin contact area; and
a lead wire connector extending outwardly from the body,
wherein each of the central cavity area and the skin contact area of the rim are electrically conductive and are located on the skin side.

2. The medical electrode of claim 1, wherein the at least one protrusion comprises a rib arranged within +/−10 degrees of a radial direction relatively to a central axis through the central cavity.

3. The medical electrode of claim 1, wherein the at least one protrusion comprises between 3 and 5 ribs, each of the ribs arranged within +/−10 degrees of a radial direction relatively to a central axis through the central cavity.

4. The medical electrode of claim 3, wherein the ribs are evenly distributed within the central area.

5. The medical electrode of claim 4, wherein each of the ribs comprises a rib area, wherein the electrode comprises a total active area comprised of the central cavity area, the rib areas, and the skin contact area, and wherein the total active area is in the range of 95 to 130 mm².

6. The medical electrode of claim 1, wherein the dome has the shape of a truncated cone with rounded edges.

7. The medical electrode of claim 1, wherein the lead wire connector comprises a depression and at least one wall adjacent to the depression.

8. The medical electrode of claim 7, the depression is sectioned by a step providing a shallow conducting portion and a deep connection portion, the deep connection portion being outward of the shallow conducting portion.

9. The medical electrode of claim 1, wherein the skin contact area of the rim comprises an outer periphery, and wherein the medical electrode further comprises cut-outs extending inwardly from the outer periphery of the rim.

10. The medical electrode of claim 9, wherein the cut-outs comprise between 3 and 5 cut-outs, each of the cut-outs having a radial depth in the range of 0.5-1.5 mm.

11. The medical electrode of claim 1, wherein the body comprises a polymer-based core material with a conductive surface coating of Ag/AgCl.

12. The medical electrode of claim 1, wherein the cavity is prefilled with a conductive gel.

13. The medical electrode of claim 1, wherein the medical electrode is an MR conditional electrode.

14. An electrode array comprising a plurality of the medical electrode of claim 1 and lead wires, each of the lead wires comprising a lead wire conductor with an electrically insulating cladding, and each lead wire connector of the plurality of the medical electrode being electrically conductive, wherein each of the lead wires has a first end and a second end, the first end being physically embedded in and electrically connected to a respective one of the lead wire connectors of the plurality of the medical electrode, and the second end being connected to an electrode connector.

15. The electrode array of claim 14, wherein the lead wire conductor of each of the lead wires is made of an electrically conductive non-magnetic material or materials.

16. The electrode array of claim 14, wherein each lead wire connector of the plurality of the medical electrode is comprised substantially of polymer materials and devoid of metal parts.

17. The electrode array of claim 14, wherein each lead wire connector of the plurality of the medical electrode has a length in the interval of 30 to 50 cm.

18. The electrode array of claim 1, wherein the plurality of the medical electrode are MR conditional.

19. A method of making the electrode array of claim 14, the method comprising, for each of the medical electrodes of the plurality of the medical electrode:
positioning the first end of the lead wire in the lead wire connector;
applying energy to material of the lead wire connectors to soften or melt the material; and
allowing the softened or molten material to set around the lead wire, thereby integrating the lead wire in the lead wire connector of the medical electrode.
